(19) United States
(12) Patent Application Publication
(10) Pub. No.: US 2023/0200845 A1
(43) Pub. Date: Jun. 29, 2023

(54) GUIDEWIRE HAVING GUIDEWIRE MARKERS

(71) Applicant: Boston Scientific Medical Device Limited, Galway (IE)

(72) Inventors: James Klein, Toronto (CA); Maria Luk, Kleinburg (CA); John Paul Urbanski, Toronto (CA)

(21) Appl. No.: 18/177,603

(22) Filed: Mar. 2, 2023

Related U.S. Application Data
(63) Continuation of application No. PCT/IB2021/058023, filed on Sep. 2, 2021.
(60) Provisional application No. 63/074,672, filed on Sep. 4, 2020.

(51) Int. Cl. A61B 17/34; A61M 25/09

(52) U.S. Cl. CPC A61B 17/34; A61M 25/09; A61B 2017/00247

(57) ABSTRACT

A guidewire for performing a transseptal puncture, the guidewire having a distal end and a proximal end with an elongated length therebetween. The guidewire has at least one recess along its elongated length. The distal end of the guidewire is configured to puncture tissue. At least one guidewire marker is configured to be securely positioned, respectively at said at least one recess of the elongated length of the guidewire.

### CROSS-REFERENCE TO RELATED APPLICATIONS:

This application is a continuation of and claims the benefit of International Application Number PCT/IB2021/058023, entitled “GUIDEWIRE HAVING GUIDEWIRE MARKERS,” and filed Sep. 2, 2021, which claims the benefit of U.S. Provisional Application No. 63/074,672, entitled “GUIDEWIRE HAVING GUIDEWIRE MARKERS,” and filed Sep. 4, 2020, which are hereby incorporated by reference in their entireties.

### TECHNICAL FIELD

This disclosure relates to the technical field of: (A) an apparatus including a guidewire having guidewire markers; and (B) an apparatus for use with guidewire markers, the apparatus including a guidewire; and (C) an apparatus for use with a guidewire, the apparatus including guidewire markers; and (D) a method associated with a guidewire; and (E) a method associated with guidewire markers; and (F) a method associated with a combination of guidewire markers and a guidewire.

### BACKGROUND

Known medical devices are configured to facilitate a medical procedure, and help healthcare providers diagnose and/or treat medical conditions of sick patients.

### SUMMARY

It will be appreciated that there exists a need to mitigate (at least in part) at least one problem associated with the existing (known) guidewires (also called the existing technology). After much study of, and experimentation with, the existing (known) guidewires, an understanding (at least in part) of the problem and its solution have been identified (at least in part) and are articulated (at least in part) as follows:

Radio frequency guidewires need relatively precise positioning of a distal electrode relative to a distal end of ancillary devices (such as, dilators, etc.) during use. Guidewires are, generally, much longer than their supporting dilators, and may not have dedicated handles located along their length. This may be true for the case where the guidewire is used to support the exchange of the ancillary devices as there is no handle, or other equivalent features, that might prevent such exchange activities. As such, positioning is done by trial and error, or with the use of external medical imaging equipment, such as fluoroscopy, etc.

A means to align a guidewire within supporting devices without requiring the use of fluoroscopy may be desirable to surgeons, and/or other users such as physicians, interventional cardiologists, etc. This might improve ease-of-use, lessen procedural time, and/or reduce potential risk (allow for relatively lower exposure to x-ray radiation associated with fluoroscopy, etc.).

It may be desirable to provide guidewire markers (bands) mounted to (positioned on the outer surface of) a guidewire; the markers may aid the process using the guidewire, such as for the alignment of the distal end of the dilator, the manner in which surgeons may utilize transseptal needles for a procedure, etc. For instance, before performing transseptal puncture with a needle, surgeons may calibrate the length of the needle relative to the dilator by inserting the needle and checking how close the needle hub is to the dilator hub once the distal end of the needle begins to protrude from the exit port of the dilator. As the distance between hubs, typically, may be measured in finger widths, surgeons sometimes referred to this arrangement as the ‘two-finger calibration technique’. When performing a transseptal puncture procedure with a guidewire, there may be no hub available with which to perform the two-finger calibration technique. However, one might simulate the hub by providing a visual indicator (a contrast indicator), such as one or more guidewire markers (also called marker bands). The surgeon might perform the same calibration by using the guidewire markers in place of the needle hub. From here, the use of the guidewire bands positioned at multiple locations may be desirable for providing more information than might be possible with utilizing a traditional needle hub by itself.

In one broad aspect of the present invention, a guidewire for performing a transseptal puncture is provided, the guidewire comprising: a distal end and a proximal end, with an elongated length therebetween, the elongated length including at least one recess being located, at least in part, therealong; the distal end configured to puncture tissue; and, at least one guidewire marker, wherein the at least one guidewire marker is configured to be securely positioned, respectively at said at least one recess of the elongated length of the guidewire.

In another broad aspect of the present invention, an assembly for performing a transseptal puncture is disclosed, the assembly comprising: a guidewire configured for puncturing tissue, the guidewire comprising: a guidewire distal end and a guidewire proximal end with an elongated length therebetween, the elongated length including at least one recess being located, at least in part, therealong; the guidewire distal end configured to puncture tissue; and, at least one guidewire marker, wherein the at least one guidewire marker is configured to be securely positioned, respectively at said at least one recess of the elongated length of the guidewire; an ancillary device comprising an ancillary device distal end and an ancillary device proximal end with a lumen for receiving the guidewire therebetween; and, wherein when the guidewire is inserted within the lumen, the at least one guidewire marker allows the guidewire to be positioned relative to the ancillary device proximal end.

In another broad aspect of the present invention, a method of using a guidewire is disclosed, the method comprising: presenting, visually, guidewire markers along the guidewire.

In another broad aspect of the present invention, a method of using a combination of guidewire markers and a guidewire having an elongated length including discrete spaced-apart positions being located, at least in part, therealong is disclosed, the method comprising: securely respectively receiving, at said discrete spaced-apart positions of the guidewire, the guidewire markers.

In another broad aspect of the present invention, A method of manufacturing a guidewire comprising at least one recess and at least one guidewire marker, securely contacting said at least one recess, the method of manufacturing comprising the steps of: forming the at least one recess on the guidewire; positioning the at least one guidewire marker in the at least one recess; and, fixing the at least one guidewire marker in the at least one recess.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1, FIG. 2 and FIG. 3 depict a perspective view (FIG. 1) and cross-sectional views (FIG. 2 and FIG. 3) of embodiments of guidewire markers and a guidewire; and

FIG. 4, FIG. 5 and FIG. 6 depict a perspective view (FIG. 4) and cross-sectional views (FIG. 5 and FIG. 6) of embodiments of the guidewire of any one of FIG. 1, FIG. 2 and/or FIG. 3; and

FIG. 7, FIG. 8, FIG. 9, FIG. 10, FIG. 11, FIG. 12 and FIG. 13 depict a perspective view (FIG. 7), cross-sectional views (FIG. 8, FIG. 9, FIG. 10 and FIG. 11) and perspective views (FIG. 12 and FIG. 13) of embodiments of the guidewire markers and the guidewire of any one of FIG. 1, FIG. 2 and/or FIG. 3; and

FIG. 14 to FIG. 17 depict a perspective view (FIG. 14) and side views (FIG. 13 to FIG. 17) of embodiments of the guidewire of any one of FIG. 1, FIG. 2 and/or FIG. 3.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

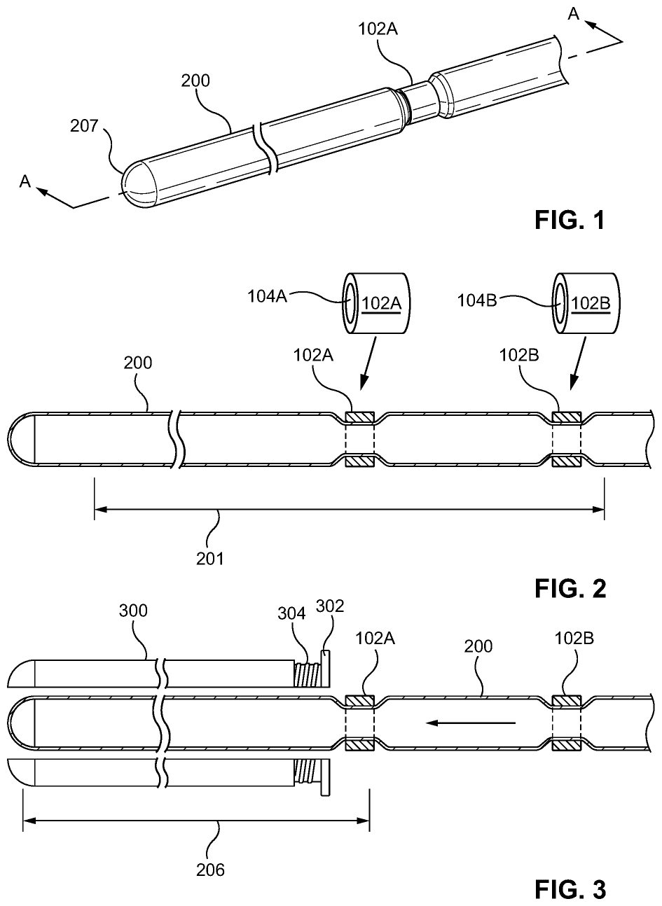

FIG. 1, FIG. 2 and FIG. 3 depict a perspective view (FIG. 1) and cross-sectional views (FIG. 2 and FIG. 3) of embodiments of guidewire markers (102A, 102B) and/or a guidewire 200. The cross-sectional views of FIG. 2 and FIG. 3 are taken along a cross-sectional line A-A extending longitudinally through the guidewire 200 of FIG. 1.

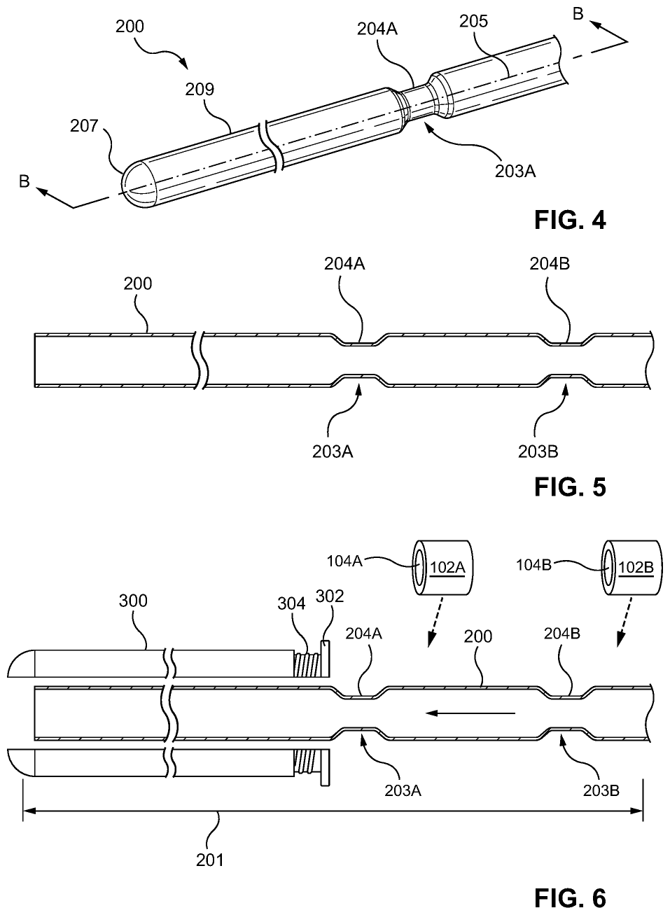

FIG. 4, FIG. 5 and FIG. 6 depict a perspective view (FIG. 4) and cross-sectional views (FIG. 5 and FIG. 6) of embodiments of the guidewire 200 of any one of FIG. 1, FIG. 2 and/or FIG. 3. The cross-sectional views of FIG. 5 and FIG. 6 are taken along a cross-sectional line B-B extending longitudinally through the guidewire 200 of FIG. 4.

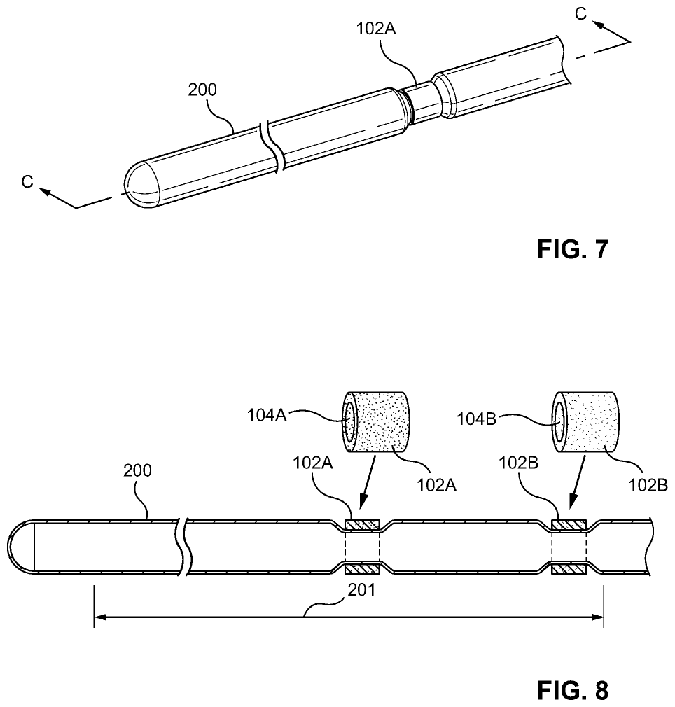

FIG. 7, FIG. 8, FIG. 9, FIG. 10, FIG. 11, FIG. 12 and FIG. 13 depict a perspective view (FIG. 7), cross-sectional views (FIG. 8, FIG. 9, FIG. 10 and FIG. 11) and perspective views (FIG. 12 and FIG. 13) of embodiments of the guidewire markers (102A, 102B) and/or the guidewire 200 of any one of FIG. 1, FIG. 2 and/or FIG. 3. The cross-sectional views of FIG. 8, FIG. 9, FIG. 10 and FIG. 11 are taken along a cross-sectional line C-C extending longitudinally through the guidewire 200 of FIG. 7.

Referring to the embodiment as depicted in FIG. 1, there is depicted an apparatus including and not limited to a guidewire 200. The guidewire 200 has (presents or visually presents) guidewire markers (102A, 102B). It will be appreciated that according to this embodiment, the guidewire 200 and the guidewire markers (102A, 102B) are provided to the end user such as a surgeon, etc. as a synergistic combination of elements. It may be considered that the synergistic combination of elements may be provided, preferably, by one (single) source and/or manufacturer.

It will be appreciated that a guidewire is not equivalent to a dilator or catheter. The guidewire does not define an elongated interior lumen extending, at least in part, along a longitudinal length of the guidewire. The guidewire has a solid core extending along the longitudinal length of the guidewire. In sharp contrast, a dilator or a catheter each defines an elongated interior lumen extending, at least in part, along a longitudinal length of the dilator or the catheter. The elongated interior lumen of the dilator or the catheter is configured to receive another medical device (such as the guidewire, etc.). Moreover, the guidewire is not configured to receive another medical device within an interior of the guidewire. It will be appreciated that the definition of inert is a substance or material that is chemically inactive, and/or a substance that is stable and does not react with other substances. The definition of ink is a wet colored fluid used for writing, drawing, printing or duplicating. The definition of printed is the production of a marking by a mechanical process involving the transfer of an image made of wet ink to a surface, and the wet ink, after application to the surface, dries. However, ink may be prone to wearing over time. In accordance with a preferred embodiment, the guidewire markers are inert, are wear resistant (at least in part) with use, degrade over time and/or are not made of ink. This arrangement, advantageously, permits cleaning and/or sanitization of the guidewire markers for redeployment in other procedures, etc. In accordance with an embodiment, the guidewire marker is encapsulated under a clear protective layer. This arrangement may include guidewire markers that are produced by ink or another thin coloured coating, as well as guidewire markers that are produced by placing a ring of material (e.g. metal for lustre, plastic for colour, etc.) around the guidewire core that may have a recesses to create space for, and ensure, no movement of the guidewire markers, or those that are generated by altering the guidewire surface directly such as through laser etching or oxidation. In accordance with an embodiment, the guidewire marker is encapsulated by an inert material and is not susceptible to damage or wear. For the case where the encapsulating inert layer is clear (light transmissive), the guidewire marker may be visible (to the user, etc.). The guidewire marker is manufactured by selective application of a thin spray-coated layer of coloured material, such as polytetrafluoroethylene (PTFE), which is then encapsulated by a thicker layer of a clear (or light transmissive) heat shrink material. While the spray-coated polytetrafluoroethylene guidewire marker might be otherwise susceptible to damage or wear (over time and/or usage), the inert clear outer layer renders the guidewire marker functionally inert. There are technical challenges associated with this method, such as the guidewire marker needing to withstand the high processing temperatures of the inert outer layer if that layer is made of PTFE heat shrink. The outer layer in this case provides electrical insulation as well as surface lubricity, etc. In accordance with an embodiment, the use of multiple segments of wire insulation to generate one or more coloured bands (for the guidewire marker). A clear layer of inert PTFE insulation may cover the guidewire markers while permitting the markers to be visible (to the user). However, guidewire markers may be generated by the colour of the PTFE coating itself. The PTFE heat shrink coating may be processed with colorants to produce a wide array of colours for the finished product. As the PTFE heat shrink coating is inert, the guidewire markers generated by alternating segments of different coloured PTFE heat shrink may constitute an inert guidewire marker. The location of the guidewire markers may be positioned to ensure a smooth transition between segments, and (preferably) ensure no gaps in the electrical insulation, thereby providing guidewire markers. If the guidewire is not configured to conduct electricity, then the guidewire insulation need not be an electrically insulative material and provide continuous insulation. However, a smooth lubricious surface may be desirable. In accordance with an embodiment, the use of an inert material on the outer surface of the guidewire may be utilized. Methods such as deposition of ink is preferably inert, particularly if they are placed on the outer surface of a PTFE layer. However, the placement of an inert materials such as another PTFE heat shrink layer or a metal ring may provide an inert guidewire marker band. The guidewire may require a local reduction in diameter (recess) at the location of the guidewire marker band so that the additional material layer avoids having a greater diameter than the rest of the guidewire. Without such a recess, the exposed edge of the guidewire marker might catch on another material and become dislodged or dislocated rendering the guidewire marker inoperative (such as no longer inert, etc.). A guidewire having guidewire markers configured to be covered with a light-transmissive coating, such as a clear coating, a transparent coating or layer. The light-transmissive coating is configured to be inert. The light-transmissive coating may be transparent or translucent to enable visualization of the marker that is covered by the light-transmissive coating. Any suitable material may be utilized for the marker (such as an non-inert material or an inert material, etc.), so long as the marker is encapsulated under the light-transmissive coating. A clear insulation layer may encapsulate/embed any material type of the band, preserving enhanced visibility of the marker while securing marker features. Further, the light-transmissive coating may be chosen to match the material substrate of the guidewire substrate. In accordance with an option, the guidewire has a nominal outer diameter, and at least one or more (preferably all of them) guidewire markers remain within the nominal outer diameter of the guidewire (that is, the one or more guidewire markers do not increase the outer diameter of the medical device). For instance, the guidewire markers include an inert guidewire marker (that is, one or more, as desired). It will be appreciated that the inert guidewire marker may include (A) a non-inert guidewire marker, and (B) an inert coating covering the non-inert guidewire marker. The guidewire has a nominal outer diameter, and the inert guidewire marker and the inert coating (covering the non-inert guidewire marker) remain within the nominal outer diameter of the guidewire.

Referring to the embodiment as depicted in FIG. 1, there is depicted a method of using the guidewire 200. The method includes and is not limited to presenting, visually, the guidewire markers (102A, 102B) along (on) the guidewire 200.

Referring to the embodiment as depicted in FIG. 1, the guidewire 200 is configured to be inserted into a confined space defined by a living body (the patient). The guidewire 200 includes (preferably) a relatively thin and flexible wire (an elongated flexible shaft) configured to be inserted into a confined or tortuous space (a confined space) defined by the living body. The guidewire 200 is (preferably) impermeable by a bodily fluid located in the confined space defined by the living body. The guidewire 200 and the guidewire markers (102A, 102B) include (in accordance with a preferred embodiment) biocompatible materials and/or properties suitable for sufficient (safe) performance (such as, dielectric strength, thermal performance, insulation and corrosion, water and heat resistance) to comply with industrial and regulatory safety standards (or compatible for medical usage). Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014]. The guidewire 200 includes, preferably, SAE (Society of Automotive Engineering) Type 304 Stainless Steel. SAE Type 304 stainless steel contains both chromium (from between about 15% to about 20%) and nickel (from between about 2% to about 10.5%) metals as the main non-iron constituents. The guidewire 200 may include (if desired) a shape-memory material configured to be manipulated and/or deformed followed by a return to the original shape that the shape-memory material was set in (prior to manipulation). Shape-memory materials (SMMs) are known and not further described in detail. Shape-memory materials are configured to recover their original shape from a significant and seemingly plastic deformation in response to a particular stimulus that is applied to the shape-memory material. This is known as the shape memory effect (SME). Superelasticity (in alloys) may be observed once the shape-memory material is deformed under the presence (an application) of a stimulus force.

Referring to the embodiment as depicted in FIG. 2, FIG. 4, FIG. 5, FIG. 6 and FIG. 8, there is provided an apparatus for use with (configured to be used with) the guidewire 200 having an elongated length 201. The apparatus includes and is not limited to the guidewire markers (102A, 102B) having the marker-contact surfaces (104A, 104B). The marker-contact surfaces (104A, 104B) are (respectively) configured to be securely positioned, respectively, at the discrete spaced-apart positions (203A, 203B). The discrete spaced-apart positions (203A, 203B) are located along, at least in part, the elongated length 201 of the guidewire 200. In some embodiments, the discrete spaced-apart positions (203A, 203B) comprise at least one recess. As an example, the recess of the guidewire 200 may be formed by having a tapered section, tapering a larger outer diameter to a smaller outer diameter. The smaller outer diameter may continue along the elongated length 201 ending at another tapered section, tapering from the smaller outer diameter to the larger outer diameter (equal to the overall outer diameter of the guidewire 200). It will be appreciated that for this embodiment, the guidewire markers (102A, 102B) are supplied, sold and/or manufactured separately from the guidewire 200; the end user may purchase these components and/or elements from separate sources (suppliers, manufacturers, etc.), if so desired.

Referring to the embodiment as depicted in FIG. 4, FIG. 5, FIG. 6 and FIG. 8, there is depicted an apparatus for use with the guidewire markers (102A, 102B). The apparatus includes the guidewire 200. The guidewire 200 has an elongated length 201 including discrete spaced-apart positions (203A, 203B) located, at least in part, therealong (along a length of the guidewire 200). The discrete spaced-apart positions (203A, 203B) configured to securely present, respectively, the guidewire markers (102A, 102B) (preferably once the guidewire markers are securely received respectively to, or mounted to, the discrete spaced-apart positions). Preferably, the discrete spaced-apart positions (203A, 203B) are configured to securely receive (securely contact), respectively, the guidewire markers (102A, 102B). It will be appreciated that for this embodiment, the guidewire 200 is supplied, sold and/or manufactured separately from the guidewire markers (102A, 102B), and the end user may purchase the elements from separate sources (suppliers, manufacturers, etc.), if so desired.

Referring to the embodiment as depicted in FIG. 4, there is provided a method for using a synergistic combination of the guidewire markers (102A, 102B) and the guidewire 200 having an elongated length 201. The elongated length 201 includes discrete spaced-apart positions (203A, 203B) located, at least in part, therealong (along the elongated length 201). The method includes and is not limited to securely receiving the guidewire markers (102A, 102B) respectively at the discrete spaced-apart positions (203A, 203B) of the guidewire 200.

Referring to the embodiment as depicted in FIG. 3, the guidewire markers (102A, 102B) are (preferably) configured to visually indicate a guidewire segment 206 of the guidewire 200.

Referring to the embodiment as depicted in FIG. 4, the guidewire 200 has (preferably) an outer surface 209 that is electrically insulated.

Referring to the embodiment as depicted in FIG. 4, the guidewire 200 has an outer surface 209 that is (configured to be) electrically insulated. The guidewire 200 includes an energy-emitting device 207. The energy-emitting device 207 is, preferably, positioned at a distal portion of the guidewire 200. It will be appreciated that other placement options for placement of the energy-emitting device 207 are possible, if so desired. An electrical wire 205 is configured to convey radio frequency energy to the energy-emitting device 207. The electrical wire 205 is, preferably, embedded within the body of the guidewire 200.

Referring to the embodiment as depicted in FIG. 4, FIG. 5 and FIG. 6, the guidewire 200 has guidewire-contact surfaces (204A, 204B) that are spaced-apart from each other. The guidewire-contact surfaces (204A, 204B) are configured to securely selectively connect to, respectively, (or securely respectively contact) the marker-contact surfaces (104A, 104B) of the guidewire markers (102A, 102B).

Referring to the embodiments as depicted in FIG. 4, FIG. 5 and FIG. 6, the guidewire markers (102A, 102B) have, respectively, marker-contact surfaces (104A, 104B). The marker-contact surfaces (104A, 104B) are configured to securely selectively connect to, respectively, (or securely respectively contact) the guidewire-contact surfaces (204A, 204B). The guidewire-contact surfaces (204A, 204B), of the guidewire 200, are spaced-apart.

Referring to the embodiments as depicted in FIG. 4, FIG. 5 and FIG. 6, the guidewire markers (102A, 102B) are positioned along an elongated length 201 of the guidewire 200. The guidewire markers (102A, 102B) are placed (spatially positioned) in a spaced-apart relationship with each other at discrete spaced-apart positions (203A, 203B).

Referring to the embodiment as depicted in FIG. 6, the guidewire 200 has an elongated length 201. The guidewire 200 includes discrete spaced-apart positions (203A, 203B) located, at least in part, therealong (along the elongated length 201). The guidewire markers (102A, 102B) are configured to be securely positioned, respectively, at the discrete spaced-apart positions (203A, 203B) of the elongated length 201 of the guidewire 200. That is, the marker 102A is to be securely positioned at the discrete spaced-apart position 203A, and the marker 102B is to be securely positioned at the discrete spaced-apart position 203B, etc.

Referring to the embodiment as depicted in FIG. 6, the guidewire 200 has the guidewire-contact surfaces (204A, 204B). The guidewire-contact surfaces (204A, 204B) are spaced apart from each other. The guidewire markers (102A, 102B) have, respectively, marker-contact surfaces (104A, 104B). The guidewire-contact surfaces (204A, 204B) are configured to securely selectively connect to, respectively, (or securely respectively contact) the marker-contact surfaces (104A, 104B) of the guidewire markers (102A, 102B). That is, the guidewire-contact surface 204A is configured to securely selectively connect to (make securely contact with) the marker-contact surface 104A, and the guidewire-contact surface 204B is configured to securely selectively connect to (make securely contact with) the marker-contact surfaces 104B, etc.

Referring to the embodiment as depicted in FIG. 6, the guidewire 200 has the guidewire-contact surfaces (204A, 204B) that are spaced apart from each other. The guidewire-contact surfaces (204A, 204B) are configured to securely selectively connect, respectively, (or securely respectively contact) the marker-contact surfaces (104A, 104B) of the guidewire markers (102A, 102B).

Referring to the embodiment as depicted in FIG. 6, the guidewire markers (102A, 102B) are positioned along the elongated length 201 of the guidewire 200. The guidewire markers (102A, 102B) are placed (located) in a spaced-apart relationship with each other at discrete spaced-apart positions (203A, 203B).

Referring to the embodiments as depicted in FIG. 4, FIG. 5 and FIG. 6, the guidewire markers (102A, 102B) may be applied or installed in (at) appropriately located or spatially positioned guidewire-contact surfaces (204A, 204B); this is done, preferably, without increasing the overall outer diameter of the guidewire 200. The marker material that may provide the greatest visibility (for brighter colours, reflective surfaces, etc., and any equivalent thereof) may introduce biocompatibility, integrity or smoothness concerns that may need to be mitigated. The guidewire markers (102A, 102B) may include, preferably, bands (banded or looped material). The guidewire-contact surfaces (204A, 204B) may include, preferably, spaced-apart troughs formed on the outer surface of the guidewire 200. Placing the guidewire markers (102A, 102B) in a recess (guidewire-contact surfaces (204A, 204B)) relative to the overall profile of the guidewire 200 may help secure the position of the guidewire markers (102A, 102B) when the guidewire 200 is advanced through an ancillary device 300, such as a sheath assembly, etc., and any equivalent thereof. The ancillary device 300 is configured to slidably receive the guidewire 200, etc. The overall guidewire diameter, located along the region of the guidewire markers (102A, 102B), may not be significantly larger compared to the remainder of the guidewire 200. Likewise, any reduction in the diameter of the guidewire 200 to accommodate (the location of) the guidewire markers (102A, 102B) may avoid the adverse effect that may result from lesser stiffness of the guidewire 200. It will be appreciated that to accommodate (the location of) the guidewire markers (102A, 102B) at the outer surface of the guidewire 200, the outer diameter of the portions (sections) of the guidewire 200 may be reduced (if desired), in which the portions are located proximate to where the guidewire markers (102A, 102B) are to be located (mounted, positioned, etc.). Therefore, mitigation may be considered or applied to these reduced portions (of the outer diameter of the guidewire 200) to reduce or avoid potential adverse effect that may result from a lower stiffness of the guidewire 200 (at the reduced portions). For the case where there is a reduction in the outer diameter (of the guidewire 200) to accommodate at least one of the guidewire markers, the guidewire stiffness may be decreased; however, since the guidewire markers are positioned at these reduced portions (local zones of reduction), the guidewire markers may counter the reduction in stiffness at the reduced portions (of the guidewire 200), or other stiffness mitigation may be taken to counter the potential lowering of stiffness.

Referring to the embodiments as depicted in FIG. 4, FIG. 5 and FIG. 6, it will be appreciated that known guidewire materials may be limited in (for) colour and/or opacity. The guidewire markers (102A, 102B) may offer (provide or display) attributes that enhance visibility for users (such as, brightness, reflectivity, etc., and any equivalent thereof). For the case where the attribute of the guidewire markers (102A, 102B) may inadvertently introduce collateral risk, appropriate mitigation may be utilized, such as integrity, biocompatibility and/or degree of sharpness, etc., and any equivalent thereof. For example, for the case where three (3) different coloured guidewire markers are desired, biocompatibility data for all three guidewire markers may be required. The requirement of extensive repeat testing for such potential risks, for each type of the guidewire markers (102A, 102B), may limit flexibility (in terms of deployment and/or production of the markers, etc.). The potential risks for guidewire markers may be mitigated by providing guidewire markers that are encapsulated with a safety coating, such as a consistent, conventional and/or transparent coating, etc., and any equivalent thereof. The guidewire markers (102A, 102B) may be configured to promote visibility added at discrete locations along the length of the guidewire 200.

Referring to the embodiments as depicted in FIG. 4, FIG. 5 and FIG. 6, the overall guidewire diameter, located along the region of the guidewire markers (102A, 102B), may not be significantly larger compared to the remainder of the guidewire 200. Any reduction in the diameter of the guidewire 200 to accommodate (the location of) the guidewire markers (102A, 102B) may avoid the adverse effect that may result from a lower degree of stiffness of the guidewire 200.

Referring to the embodiments as depicted in FIG. 4, FIG. 5 and FIG. 6, the guidewire 200 has (present or visually presents) at least one or more of the guidewire markers (102A, 102B), which are, preferably positioned, or located, at a meaningful (predetermined) locations along a longitudinal length of the guidewire 200. At least one or more guidewire markers (102A, 102B) may be, for instance, visually distinct (may have or present a distinctive sensed or detectable attribute) from the remainder of the guidewire 200. The guidewire markers (102A, 102B) may have clear (sharp) edges so that it may be relatively easier to view where the guidewire markers (102A, 102B) begin and end (distinctive edges or boundaries). In other words, the guidewire markers (102A, 102B) comprise edges which are clearly pronounced (evident) such that the user is able to clearly see the guidewire markers (102A, 102B) boundaries, This may be achieved by using a solid, brightly coloured, polymer ring, such as a heat shrink tubing, or a metal ring that is swaged into place, etc., and any equivalent thereof. Other methods of producing the guidewire marker (102A, 102B) may include ink deposition, spray coating, or selective coating removal, which may also be effective and might need to be tightly controlled so as to not result in faded in edges of the guidewire markers (102A, 102B), etc.

Referring to the embodiments as depicted in FIG. 4, FIG. 5 and FIG. 6, the location of the guidewire marker (102A, 102B) may be tightly controlled and immobile. To this end, the guidewire marker (102A, 102B) may include a solid material and may be placed in a recess, positioned or formed in the outer surface of the profile of the guidewire 200, thereby ensuring that the guidewire marker (102A, 102B) is held in a relatively secure position within the recess.

Referring to the embodiments as depicted in FIG. 4, FIG. 5 and FIG. 6, the thickness of the guidewire marker (102A, 102B) may be minimized so that the outer surface of the finished guidewire 200 is smooth once combined with the guidewire marker (102A, 102B). The impact of material thickness may be mitigated by placing the guidewire marker (102A, 102B) in a recess formed in the outer surface of the profile of the guidewire 200. Minimizing the thickness of the guidewire marker (102A, 102B), such as by using thin-walled heatshrink (etc.), may reduce the necessary depth of the recesses, which may thereby reduce the impact to the mechanical performance of the guidewire 200.

Referring to the embodiments as depicted in FIG. 4, FIG. 5 and FIG. 6, the external coating over the guidewire 200 may, preferably, not obscure the guidewire marker (102A, 102B). In accordance with a preferred embodiment, the coating may be transparent so that there is no reduction in the visibility of the guidewire marker (102A, 102B). However, some visibility reduction may be acceptable as long as the guidewire marker (102A, 102B) remains visible (detectable) in the lighted conditions of the room (such as a catheter lab, etc.). The coating may be smooth and lubricious for ease of insertion through vasculature and other devices, and may be, preferably, biocompatible. For the case where the guidewire 200 may be insulated, it may be advantageous for the coating (outer layer) to also provide electrical insulation (as depicted in FIG. 4). The outer coating may maintain integrity throughout expected handling and manipulation of the guidewire 200, and may not produce particles that could be released into the bloodstream of the patient. The coating may also be nonporous so that materials beneath the coating are not blood contacting, as this may allow for more freedom in material selection for the guidewire markers (102A, 102B) including colorants, etc. A single continuous single layer of PTFE (polytetrafluoroethylene) coating a length of the guidewire 200, including any guidewire markers (102A, 102B), may accomplish these requirements.

Referring to the embodiments as depicted in FIG. 4, FIG. 5 and FIG. 6, the guidewire markers (102A, 102B) may include at least one, or more, visual indicators (contrast indicators), such as a colour and/or a pattern (visual pattern and/or tactile pattern, etc.). The visual indicator may be configured to correspond to predetermined information (such as distance from the marker to the tip of the guidewire, etc.). The visual indicator may be selected to provide traffic light colors (such as, red may indicate a stop condition, green may indicate a go condition, yellow may indicate a standby condition, etc.). The visual indicator may provide patterns that match or correspond to specific measured positions (e.g. five (5) stripes for about a five (5) millimeter length from the distal tip of the guidewire 200, two (2) stripes indicating a two (2) millimeter distance from the distal tip of the guidewire 200, etc.).

Referring to the embodiments as depicted in FIG. 3, FIG. 6, FIG. 9 and FIG. 10, the visual indicator (contrast indicator, patterns and/or colours) used in the guidewire markers (102A, 102B) may be matched to the ancillary-device marker 302 positioned on the hub 304 of an ancillary device 300 (such as, a dilator assembly, etc.). This is done such that the ancillary-device marker 302 and the guidewire markers (102A, 102B) are aligned, the user may know (be confident) that the tips of both the guidewire 200 and the ancillary device 300 are also aligned (with each other).

Referring to the embodiments as depicted in FIG. 4, FIG. 5 and FIG. 6, a local reduction (such as a recess) formed the outer diameter of the guidewire 200 may be implemented, and may provide tactile feedback; it will be appreciated that such visual and/or tactile features may be difficult to detect (such as in rooms with dim lighting conditions). Likewise, the ability to denote multiple positions of interest may be challenging without differentiating colours, arrangements or other visible features.

Referring to the embodiments as depicted in FIG. 4, FIG. 5 and FIG. 6, the guidewire markers (102A, 102B) may include any material provided that the guidewire markers (102A, 102B) are encapsulated under a clear protective coating (biocompatible coating).

Referring to the embodiments as depicted in FIG. 4, FIG. 5 and FIG. 6, the guidewire-contact surfaces (204A, 204B) may include recesses formed in the outer surface (outer profile) of the guidewire 200. Recesses (formed on the outer diameter of the guidewire 200) may help control the location of the guidewire markers (102A, 102B). The required depth of the recesses may depend on the thickness of the material of the guidewire markers (102A, 102B). For the case where the material used in the guidewire markers (102A, 102B) is thin enough to be negligible to impact the finished surface profile of the guidewire 200, the guidewire-contact surfaces (204A, 204B) associated with the recesses (as depicted) may be unnecessary (and not utilized); that is, the recesses may be optional provided that the guidewire markers (102A, 102B) merely add a negligible thickness (a relatively minor amount) to the outer diameter so of the guidewire 200.

Referring to the embodiments as depicted in FIG. 4, FIG. 5 and FIG. 6, the visual indicators (contrast indicators, colours and or patterns) of the guidewire markers (102A, 102B) may provide visual contrast against the rest of the guidewire 200; therefore, multiple colours or patterns may not be necessary. Multiple colours or patterns may be selected if each one of the guidewire markers (102A, 102B) is meant to correspond to a different position (along the guidewire 200). However, this may not be necessary as the user may differentiate the guidewire markers (102A, 102B) by their relative position on the length of the guidewire 200. For the case where visual indicators, colours and/or patterns (and any equivalent thereof) are deployed on (to or with) the guidewire markers (102A, 102B), any type of visual indicator may be chosen, as long as they are visually distinct (from each other and/or from the guidewire 200).

Referring to the embodiments as depicted in FIG. 4, FIG. 5 and FIG. 6, the guidewire marker (102A, 102B) may include tactile indicators (for touch sensing by the user), so that the user need not look away from the imaging screens (displays) of medical-imaging equipment; it will be appreciated that a guidewire marker (102A, 102B) having tactile indicators might be misconstrued as defective and/or as the guidewire 200 getting inadvertently caught during a procedure.

Referring to the embodiments as depicted in FIG. 4, FIG. 5 and FIG. 6, the guidewire marker (102A, 102B) may include oxidized markings formed directly on (to) the outer surface of the guidewire 200, so that the material for the guidewire marker (102A, 102B) is not needed per se, though this arrangement might limit the available marker colours, etc.

Referring to the embodiments as depicted in FIG. 4, FIG. 5 and FIG. 6, the guidewire marker (102A, 102B) may include etch or print markings formed on the outer surface of the guidewire 200 and/or an insulative coating formed on the guidewire 200, so that the coating need not be of a clear material per se (though this arrangement might limit the achievable contrast). In addition, the guidewire markers (102A, 102B) includes materials, preferably, that do not wear down with use and/or degrade over time.

Referring to the embodiments as depicted in FIG. 4, FIG. 5 and FIG. 6, the guidewire marker (102A, 102B) may include a heatshrink material, preferably with a spiral pattern as the coating so that the user has visual cues to the speed at which the guidewire 200 is being inserted and/or retracted, though this arrangement does not provide discrete locations of the guidewire markers (102A, 102B).

Referring to the embodiments as depicted in FIG. 4, FIG. 5 and FIG. 6, the guidewire 200 may be configured to provide two (2) colours formed on the outer surface of the guidewire 200, such that the border between the two adjacently positioned colours provides the guidewire markers (102A, 102B); for instance, a blue coloured distal portion of the guidewire 200 (for one of the indicated positions), and a red coloured proximal portion of the guidewire 200 (for another of the indicated positions). This might provide high visual contrast, but may be difficult to interpret as multiple colour-change locations being located too close together and might look like the another one of the guidewire markers (102A, 102B), etc.

Referring to the embodiments as depicted in FIG. 4, FIG. 5 and FIG. 6, the user may use imaging techniques (e.g. fluoroscopy) to directly observe the relative position of distal ends of the guidewire 200 and the ancillary device 300, so long as both devices are visible under the imaging method. However, the guidewire markers (102A, 102B) may provide functionality without medical imaging equipment (as it may be desirable to reduce exposure to associated radiation to the surgeon, etc.).

Referring to the embodiments as depicted in FIG. 4, FIG. 5 and FIG. 6, the guidewire 200 may be provided with a removable handle that is preloaded on the guidewire 200 into position such that when the handle meets the dilator hub, the guidewire 200 is positioned in a puncture position. The handle may then be detached to allow for device exchanges. This arrangement might be severely limited as it may only work as long as the detachable handle is not moved. Preloading the handle on the guidewire 200 may present challenges for guidewire packaging and/or dispensing, as well as sterilization, etc.

Referring to the embodiments as depicted in FIG. 4, FIG. 5 and FIG. 6, other methods of indicating alignment between the guidewire 200 and the ancillary device 300 that do not rely on imaging may include removing the guidewire insulation at a strategic location of the guidewire 200 that aligns with an electrical contact located on a dilator when the guidewire 200 is positioned in a puncture position, etc.

Referring to the embodiments as depicted in FIG. 4, FIG. 5 and FIG. 6, the guidewire 200 includes an energy device (such as, the PowerWire (TRADEMARK) RF guidewire, manufactured by the BAYLIS MEDICAL COMPANY located in Toronto, Canada). The guidewire 200 may also be integrated with (used with) the energy generator (known and not depicted); an alert may be sounded when the guidewire 200 is placed in position (in the patient) and/or when radio frequency energy is applied once the guidewire 200 is ready to be activated. This arrangement may help balance the freedom available to the surgeons when they think it is most appropriate to apply radio frequency energy in view of any potential for current leakage risks, etc.

Referring to the embodiments as depicted in FIG. 4, FIG. 5 and FIG. 6, the outer surface of the guidewire 200 may include a feature configured to lock (mechanically) with a corresponding feature on a dilator hub when the guidewire 200 is inserted to a puncture position. The lock may then be released to allow for device exchanges. For the case where multiple distance positions (lengths) for the guidewire 200 are desired, the guidewire 200 may include several instances of the guidewire markers (102A, 102B) positioned along the length of the guidewire 200 (that is, each guidewire marker may indicating a predetermined corresponding guidewire insertion depth, etc.). This arrangement may be limited as any mechanical locking feature should not diminish (negatively impact) the smooth, lubricious nature of the guidewire 200 and/or alter the compatibility of the guidewire 200 with other exchange devices, etc.

Referring to the embodiments as depicted in FIG. 4, FIG. 5 and FIG. 6, recesses are formed or created in the outer profile of the guidewire 200, to accommodate for the thickness of the guidewire markers (102A, 102B) which may result in a localized decrease in the guidewire stiffness. This arrangement may lead to an increased risk of kinking in the guidewire 200. This condition may be mitigated by increasing the transition (the taper section) length on either end of the recess. While this arrangement may reduce the impact, a residual risk may remain of kinking in the guidewire 200. It may be preferred to not form the recesses (grooves or troughs), and this may limit the types (configurations) of the guidewire markers (102A, 102B) to those of negligible thickness. Guidewire markers (102A, 102B) may be produced by spray coating the length of the guidewire 200 and by selectively removing the spray coating to reveal contrasting surface sections (bands, portions, etc.) on the outer diameter of the guidewire 200, and/or by applying a contrasting spray coating to produce the guidewire marker (102A, 102B) in a direct fashion. In both cases, the spray coatings may be encapsulated under the clear layer of PTFE coating that forms an insulation layer (if desired). While these method may limit the colours available as the spray coating may be durable to withstand high processing temperatures, they may be effective in producing (forming) the guidewire markers (102A, 102B) at tightly controlled locations with sufficient visual clarity.

Referring to the embodiments as depicted in FIG. 6 and FIG. 9, the location of the guidewire markers (102A, 102B) may correspond with the hub length of the ancillary device 300 (such as a dilator). As well, various colours and/or pattern arrangements may be used to indicate functional positions of the guidewire 200 (for example, compatibility when corresponding to different lengths of catheters, or a status indicator to indicate “too distal”, “position ok”, “position too far”, etc.).

Referring to the embodiments as depicted in FIG. 7, FIG. 8, FIG. 9 and FIG. 10, the guidewire markers (102A, 102B) are configured to indicate depth once the guidewire marker (102A, 102B) reaches an entrance of (or exit of) the ancillary device 300, etc.

Referring to the embodiments as depicted in FIG. 7, FIG. 8, FIG. 9 and FIG. 10, the guidewire markers (102A, 102B) may have different visual effects, such as colours and/or textural arrangements, configured to indicate compatibility with the ancillary device 300. That is, if it is expected that the guidewire 200 may be used with different types of dilators of different lengths, and the user may need to know how far to insert the guidewire 200 before the distal ends of the guidewire 200 and the ancillary device 300 are aligned, multiple guidewire markers (102A, 102B) may deployed to the guidewire 200 with each corresponding to the length of a given dilator model. If the colour or arrangement of the guidewire markers (102A, 102B) matches a similar mark on the hub of the dilator, the physician may know that when the guidewire markers (102A, 102B) are aligned with the matching hub, the distal ends of both the guidewire 200 and the ancillary device 300 are also aligned.

Referring to the embodiments as depicted in FIG. 7, FIG. 8, FIG. 9 and FIG. 10, the guidewire markers (102A, 102B) may provide information about multiple positions associated with geometric aspects of the guidewire 200 (relative to other equipment, such as ancillary devices). Examples of useful positions may include when the distal tip of the guidewire 200 is retracted inside the dilator, when the distal tip of the guidewire 200 is protruding enough to perform a puncture, when the distal tip of the guidewire 200 is protruding too far to perform a puncture, and/or when the guidewire 200 has been deployed far enough such that the maximum rail is provided at the septum to support crossing with ancillary devices (known and not depicted). To this end, multiple guidewire markers (102A, 102B) may be provided corresponding to each of these positions.

Referring to the embodiments as depicted in FIG. 7, FIG. 8, FIG. 9 and FIG. 10, for the case where the guidewire markers (102A, 102B) vary in appearance around the circumference of the guidewire 200, they may be used to provide rotational position information in addition to axial position information. This arrangement may be used to provide information about the rotational position of the tip of the guidewire 200, which may be particularly helpful in knowing which direction the guidewire 200 may point when the guidewire 200 becomes deployed following transseptal puncture. For example, two-colour guidewire markers (102A, 102B) (e.g. one red and the other white) might be provided so that when the red marker is visible, the tip (such as a J tip) of the guidewire 200 may curve down and toward the ventricle when deployed. When the white marker is visible, the tip of the guidewire 200 may curve up and away from the ventricle when deployed. This might be a useful aid particularly where the anatomy is irregular as it may be desirable to avoid interaction between the tip of the guidewire 200 and structures of the heart.

Referring to the embodiments as depicted in FIG. 7, FIG. 8, FIG. 9 and FIG. 10, it may not be necessary for corresponding visual indicators (contrast indicators, colours and/or patterns, tactile formations, etc.) to be used on compatible devices, as there are other means by which the user may confirm that the guidewire markers (102A, 102B) correspond to the device in use (e.g. fluoroscopic imaging). If matching visual indicators are required and are to be placed on compatible devices, any type of visual indicator may be selected as long as the visual indicator is visually and/or tactilely recognizable as that positioned on the guidewire 200.

Referring to the embodiment as depicted in FIG. 11, a coating material 500 is applied to the guidewire markers (102A, 102B), and is, preferably, biocompatible. It is preferred that the coating material 500 applied to (such as the outer surfaces) of the guidewire markers (102A, 102B) have electrical insulation properties, such as polytetrafluoroethylene (PTFE), etc., and any equivalent thereof. The coating material 500 may be transparent and/or translucent (light transmissive) to enable visualization of the encapsulated guidewire markers (102A, 102B). For improved integrity, the coating material 500 may be well adhered to the base material of the guidewire markers (102A, 102B), etc.

Referring to the embodiment as depicted in FIG. 11, the coating material 500 may include a clear insulation, and/or may encapsulate any type of material, preserving the enhanced visibility features of the guidewire markers (102A, 102B). Further, the coating material 500 may be chosen to match the substrate material of the guidewire 200. The guidewire markers (102A, 102B) may feature an exotic surface (such as, reflective metallic coils, dimpled bands, knurls, etc.). The exotic surface is configured to promote visibility from wider viewing angles; these types of treatments might adversely interact with tissue or ancillary devices. A clear coating overtop secures the feature of the guidewire markers (102A, 102B), and ensures a consistent outer material that interacts with tissue and catheters as safely as the base material of the guidewire 200. The coating material 500 may include a clear coating placed over the guidewire markers (102A, 102B). The coating material 500 may not be necessary if the guidewire markers (102A, 102B) satisfy the requirements (for material safety, bio-compatibility, etc.), such as allowing the overall length of the guidewire 200 to have a smooth surface of constant diameter, that the guidewire 200 be sufficiently insulated, that any surface materials are biocompatible, and/or that the surface of the guidewire 200 be smooth and lubricious, etc. This arrangement may be achieved by using guidewire markers (102A, 102B) that are biocompatible, are insulative, and/or have relatively equal thickness to the coating on the rest of the guidewire 200, etc. A discontinuity between the guidewire markers (102A, 102B) and the coating material 500 may result in unacceptable (electrical) current leakage, and a smooth continuous coating over a length of the guidewire 200 may mitigate (at least in part) the effects resulting from the discontinuity, etc. While PTFE may be a desirable material choice, the coating material 500 may be of any type of material or have any type of suitable attribute including clear, lubricious, biocompatible, and/or electrically insulative (in any combination and/or permutation thereof), etc, depending on the purpose.

Referring to the embodiment as depicted in FIG. 12, at least one of the guidewire markers (102A, 102B) includes a layer (of material) deposited on, or applied to, the outer surface of the guidewire 200. The layer of material may include a heat shrinkable tube, etc., and any equivalent thereof (as previously described). The layer provides a distinctive pattern (tactile or visual) in comparison to the outer surface of the guidewire 200. The layer, preferably, does not add a significant amount to the nominal outer diameter of the guidewire 200.

Referring to the embodiment as depicted in FIG. 13, at least one of the guidewire markers (102A, 102B) includes a relief pattern formed on (etched onto) the outer surface of the guidewire 200 (as previously described). The relief pattern provides a distinctive pattern (tactile or visual) in comparison to the outer surface of the guidewire 200.

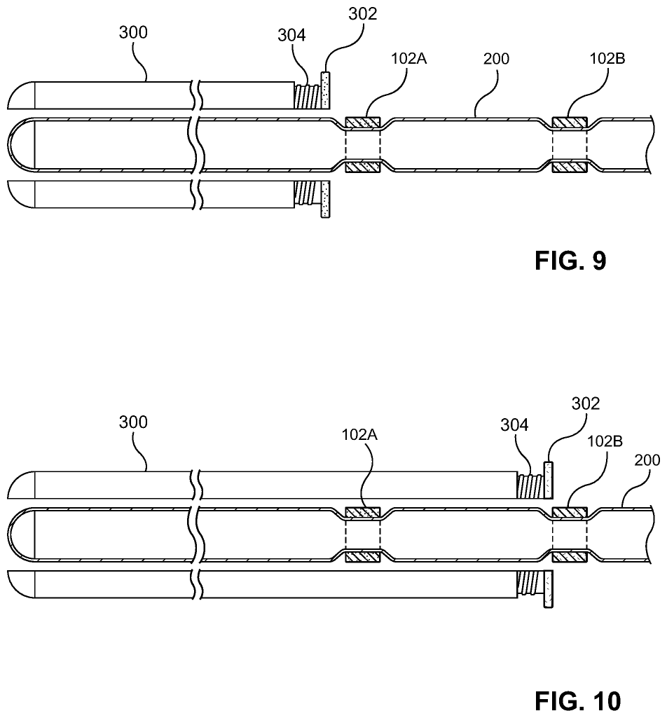

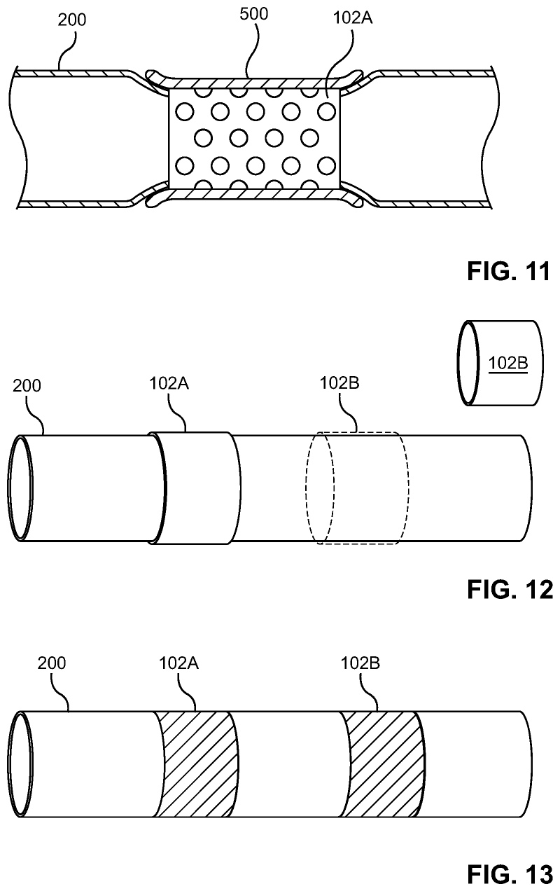

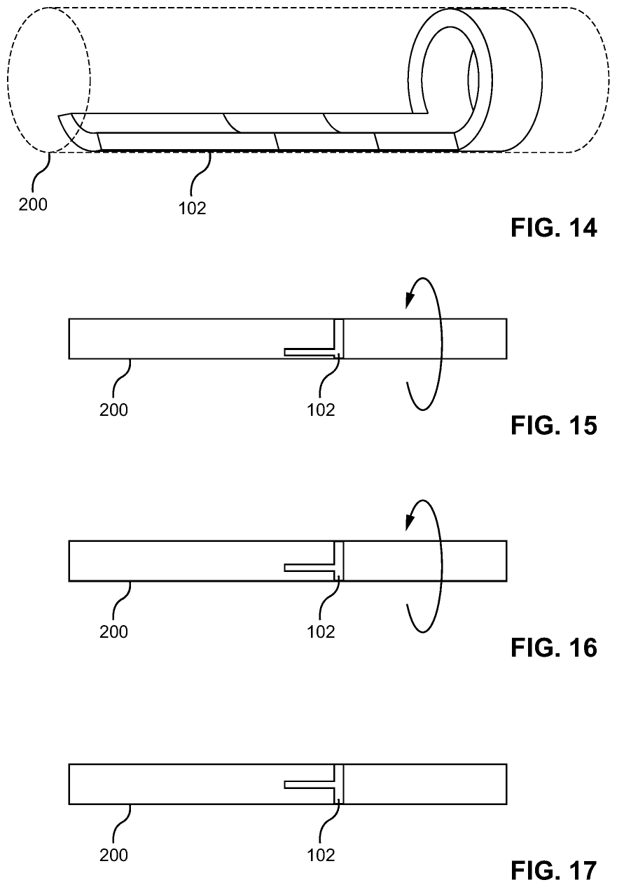

FIG. 14 to FIG. 17 depict a perspective view (FIG. 14) and side views (FIG. 13 to FIG. 17) of embodiments of the guidewire of any one of FIG. 1, FIG. 2 and/or FIG. 3.

Referring to the embodiment as depicted in FIG. 14, the inert guidewire marker 102 forms (includes) a ring-shaped formation having an elongated stem extending from the ring-shaped formation. The elongated stem is positioned on the outer surface of the guidewire 200, and is aligned axially along the elongated length of the guidewire 200 along the outer diameter of the guidewire 200. The ring-shaped formation is positioned around the outer circumference (diameter) of the guidewire 200.

Referring to the embodiment as depicted in FIG. 15, the guidewire 200 is rotated along its longitudinal axis, revealing at least part of the elongated stem extending from the ring-shaped formation; as depicted in FIG. 15., the inert guidewire marker 102 is viewed by the user as an L-shaped formation as a result of the depicted rotational orientation of the guidewire 200.

Referring to the embodiment as depicted in FIG. 16, the guidewire 200 is further rotated along its longitudinal axis, revealing more of the elongated stem extending from the ring-shaped formation; as depicted in FIG. 16., the inert guidewire marker 102 is viewed by the user as an odd shaped (unsymmetrical) T-shaped formation as a result of the depicted rotational orientation of the guidewire 200.

Referring to the embodiment as depicted in FIG. 17, the guidewire 200 is rotated along its longitudinal axis, further revealing the elongated stem extending from the ring-shaped formation; as depicted in FIG. 17, the inert guidewire marker 102 is viewed by the user as a symmetrical T-shaped formation as a result of the depicted rotational orientation of the guidewire 200. The guidewire marker 102 is configured to be viewable by a medical imaging system. When viewed under a medical imaging system, the guidewire marker 102 may appear as a T-shaped formation (ad depicted in FIG. 17), and when viewed (under medical imaging) from the side, the guidewire marker 102 appears as an L-shape. For this case, the guidewire marker 102 may be called an LT marker. The marker backbone has a curved configuration for corresponding with a side wall of the guidewire marker 102. The guidewire marker 102 has a generally circular cross section which substantially corresponds with a cross section of the side wall of the guidewire marker 102 for embedding the guidewire marker 102 within the side wall of the guidewire 200.

The guidewire 200 is preferably smooth, lubricious, electrically insulating, and provide users with high visibility of the guidewire markers (102A, 102B). In some instances, these properties are achieved by applying an outer layer overtop of the outer surface 209. There currently exists challenges associated with the application of material to this outer layer, such as difficulty adhering material to the lubricious coating and/or altering the surface material properties. Furthermore, any material applied to the outer layer may be susceptible to movement as the material shrinks or relaxes over time. To overcome such challenges, the application of material underneath the outer layer allows the guidewire 200 to retain the surface's smoothness, lubricity, and insulative properties.

In some embodiments of the present invention, discrete spaced-apart positions (203A, 203B) are formed as recesses. The reduction in the outer diameter of the guidewire 200 may be achieved using known manufacturing techniques, such as centerless grinding. An advantage provided through this method is that the recess portions can be spatially positioned at a higher degree of accuracy, compared to other guidewire marker designs.

In some embodiments, the guidewire markers (102A, 102B) may be comprised of a ring, wherein the marker (102A, 102B) must have an inner diameter that is larger than the outer diameter of the guidewire 200 such that the markers (102A, 102B) may be loaded into place. Once in place, the markers (102A, 102B) may be fixed in the desired position.

In embodiments, wherein the markers (102A, 102B) are comprised of a metallic material, fixation to the guidewire 200 may include using an adhesive. The use of an adhesive may simplify the manufacturing process. In a preferred embodiment, the markers (102A, 102B) may be mechanically fixed in place through a process which will reduce the diameter, seating it within the recesses. In one example, the markers (102A, 102B) may be swaged to be fixed within the at least one recess; this process would reduce the outer diameter of the markers (102A, 102B) such that it fits within the recesses, without causing an increase in diameter of the guidewire 200. In other words, the outer diameter of the markers (102A, 102B) would be swaged to be substantially equivalent to the outer diameter of the guidewire 200. In an alternative embodiment, the markers (102A, 102B) may undergo crimping once positioned within the recesses. In another embodiment, wherein the markers (102A, 102B) are comprised of a polymer material, fixation to the guidewire 200 may be achieved using an adhesive. Alternatively, the markers (102A, 102B) may be fixed using heat shrinking. This is particularly effective if the markers (102A, 102B) are configured to be a thin-walled polymer, such as polyethylene terephthalate (PET).

Mechanically fixing the markers (102A, 102B) prevents the need to rely on the stability of the adhesive over processing, storage, or usage condition. Additionally, using a mechanical fixing technique would avoid any visual impacts from the adhesive material.

In some embodiments, the markers (102A, 102B) may be configured to comprise a surface texture. Various methods may be used to create irregularities in the surface of the markers (102A, 102B), for example laser etching, grinding, abrasive blasting may be used. Specifically, for markers (102A, 102B) comprised of a metallic material, dimpling or knurling may be employed to create a surface texture. Introducing surface irregularities/textures to the markers (102A, 102B) may ease visualization during use.

By appropriately positioning the markers (102A, 103B) in the recesses formed along the guidewire, the markers (102A, 102B) can be applied without resulting in an increase in overall diameter of the guidewire; thus, the guidewire 200 is able to retain the smooth, lubricious, and insulative properties of the outer surface 209.

Once the markers (102A, 102B) are fixed in the recesses, a clear insulative coating, as previously described above, may be applied to encapsulate the markers (102A, 102B), preserving visibility while securing the markers (102A, 102B) in place.

In some instances, the markers (102A, 102B) may be placed directly onto the core wire of the guidewire 200 (under the insulative layer) through various means such as centerless grinding of the core wire, and/or by marking the core wire directly through spray coating, and/or laser etching. Using this method, the markers (102A, 102B) would have minimal thickness and may not require at least one recess. However, this method may require additional controls such as fixing and/or masking the core wire to ensure that the markers (102A, 102B) are located at the correct position and to ensure that the edges of the markers (102A, 102B) are clearly defined. By manufacturing recesses along the guidewire 200, the markers (102A, 102B) can be placed accurately without the need of additional controls to ensure proper placement.

### EXAMPLES

1. A guidewire for performing a transseptal puncture, the guidewire comprising:
   a distal end and a proximal end with an elongated length therebetween, the elongated length including at least one recess being located, at least in part, therealong;
   the distal end configured to puncture tissue; and,
   at least one guidewire marker, wherein the at least one guidewire marker is configured to be securely positioned, respectively at said at least one recess of the elongated length of the guidewire.
2. The guidewire of example 1, wherein the guidewire has a nominal outer diameter and the at least one or more guidewire markers remain within the nominal outer diameter of the guidewire.
3. The guidewire of any one of examples 1 or 2, wherein the at least one guidewire marker is visually distinct from the guidewire.
4. The guidewire of any one of examples 1 to 3, wherein the distal end comprises an energy emitting device.
5. The guidewire of any one of examples 1 to 4, wherein the guidewire further comprises an outer layer that is electrically insulating.
6. The guidewire of example 5, wherein the outer layer encapsulates the at least one guidewire marker.
7. The guidewire of any one of examples 5 to 6, wherein the outer layer is a clear, insulating layer.
8. The guidewire of any one of examples 1 to 7, wherein the at least one guidewire marker is an inert guidewire marker.
9. The guidewire of example 8, wherein the inert guidewire marker is composed of a non-inert guidewire marker comprising an inert coating over the non-inert guidewire marker.
10. The guidewire of any one of examples 1 to 9, wherein the at least one guidewire marker further comprises an exotic surface configured to promote visibility.
11. The guidewire of example 10, wherein the exotic surface comprises reflective metallic coils.
12. The guidewire of example 10, wherein the exotic surface comprises dimple bands.
13. The guidewire of example 10, wherein the exotic surface comprises knurls.
14. The guidewire of any one of examples 1 to 13, wherein the at least one guidewire marker comprises marker contact surfaces and wherein the at least one recess comprises a guidewire contact surface and wherein the guidewire contact surface is configured to securely contact the marker contact surface.
15. The guidewire of any one of examples 1 to 14, wherein the at least one recess comprises two or more recesses and wherein the two or more recesses are spaced apart from one another along the elongated length of the guidewire.
16. The guidewire of example 15, wherein the two or more recesses form a pattern along the elongated length.
17. The guidewire of any one of examples 1 to 16, wherein the at least one guidewire marker indicates rotational positioning of the guidewire.
18. The guidewire of any one of examples 1 to 17, wherein the at least one guidewire marker mitigates the reduction in stiffness of the at least one recess.
19. The guidewire of any one of examples 1 to 18, wherein the at least one guidewire comprises sharp edges.
20. An assembly for performing a transseptal puncture, the assembly comprising:
   a guidewire configured for puncturing tissue, the guidewire comprising:
      a guidewire distal end and a guidewire proximal end with an elongated length therebetween,
      the elongated length including at least one recess being located, at least in part, therealong;
      the guidewire distal end configured to puncture tissue; and,
      at least one guidewire marker, wherein the at least one guidewire marker is configured to be securely positioned, respectively at said at least one recess of the elongated length of the guidewire;
   an ancillary device comprising an ancillary device distal end and an ancillary device proximal end with a lumen for receiving the guidewire therebetween; and, wherein when the guidewire is inserted within the lumen, the at least one guidewire marker allows the guidewire to be positioned relative to the ancillary device proximal end.
21. The assembly of example 20, wherein the proximal end of the ancillary device comprises an ancillary device marker.
22. The assembly of any one of examples 20 to 21, wherein alignment of the ancillary device proximal end and the at least one guidewire marker indicates alignment of the ancillary device distal end and the guidewire distal end.
23. The assembly of any one of examples 20 to 21, wherein alignment of the ancillary device proximal end and the at least one guidewire marker indicates the guidewire distal end is in a position to puncture.
24. The assembly of any one of examples 20 to 23, wherein the ancillary device is a dilator.
25. The assembly of any one of examples 20 to 24, wherein the guidewire has a nominal outer diameter and the at least one or more guidewire markers remain within the nominal outer diameter of the guidewire.
26. The assembly of any one of examples 20 to 25, wherein the at least one guidewire marker is visually distinct from the guidewire.
27. The assembly of any one of examples 20 to 26, wherein the guidewire distal end comprises an energy emitting device.
28. The assembly of any one of examples 20 to 27, wherein the guidewire further comprises an outer layer that is electrically insulating.
29. The assembly of example 28, wherein the outer layer encapsulates the at least one guidewire marker.
30. The assembly of any one of examples 28 to 29, wherein the outer layer is a clear, insulating layer.
31. The assembly of any one of examples 20 to 30, wherein the at least one guidewire marker is an inert guidewire marker.
32. The assembly of example 31, wherein the inert guidewire marker is composed of a non-inert guidewire marker comprising an inert coating over the non-inert guidewire marker.
33. The assembly of any one of examples 20 to 32, wherein the at least one guidewire marker further comprises an exotic surface configured to promote visibility.
34. The assembly of example 33, wherein the exotic surface comprises reflective metallic coils.
35. The assembly of example 33, wherein the exotic surface comprises dimple bands.
36. The assembly of example 33, wherein the exotic surface comprises knurls.
37. The assembly of any one of examples 20 to 36, wherein the at least one guidewire marker comprises marker contact surfaces and wherein the at least one recess comprises a guidewire contact surface and wherein the guidewire contact surface is configured to securely contact the marker contact surface.
38. The assembly of any one of examples 20 to 37, wherein the at least one recess comprises two or more recesses and wherein the two or more recesses are spaced apart from one another along the elongated length of the guidewire.
39. The assembly of example 38, wherein the two or more recesses form a pattern along the elongated length.
40. The assembly of any one of examples 20 to 39, wherein the at least one guidewire marker indicates rotational positioning of the guidewire.
41. The assembly of any one of examples 20 to 40, wherein the at least one guidewire marker mitigates the reduction in stiffness of the at least one recess.
42. The assembly of any one of examples 20 to 41, wherein the at least one guidewire comprises sharp edges.
43. A method of using a guidewire, the method comprising:
   presenting, visually, guidewire markers along the guidewire.
44. A method of using a combination of guidewire markers and a guidewire having an elongated length including discrete spaced-apart positions being located, at least in part, therealong, the method comprising:
   securely respectively receiving, at said discrete spaced-apart positions of the guidewire, the guidewire markers.
45. A method of manufacturing a guidewire comprising at least one recess and at least one guidewire marker, securely contacting said at least one recess, the method of manufacturing comprising the steps of:
   forming the at least one recess on the guidewire;
   positioning the at least one guidewire marker in the at least one recess; and,
   fixing the at least one guidewire marker in the at least one recess.
46. The method of example 45, wherein forming the at least one recess is formed on a proximal portion of the guidewire.
47. The method of any one of examples 45 to 46, wherein the method comprises an additional step of applying an outer layer overtop the guidewire, encapsulating the at least one guidewire marker.
48. The method of any one of examples 45 to 47, wherein fixing the at least one guidewire marker involves using an adhesive.
49. The method of any one of examples 45 to 47, wherein fixing the at least one guidewire marker involves swaging the at least one guidewire marker.
50. The method of any one of examples 45 to 47, wherein fixing the at least one guidewire marker involves crimping the at least one guidewire marker.
51. The method of any one of examples 45 to 50, wherein the method comprises a step of applying surface irregularities to the at least one guidewire marker.
52. The method of example 51, wherein applying surface irregularities involves laser etching.
53. The method of example 51, wherein applying surface irregularities involves abrasive blasting.
54. The method of example 51, wherein applying surface irregularities involves grinding.
55. The method of example 51, wherein applying surface irregularities involves dimpling.
56. The method of example 51, wherein applying surface irregularities involves knurling.
57. The method of any one of examples 45 to 56, wherein forming the at least one recess involves reducing an outer diameter of the guidewire.
58. The method of any one of examples 45 to 57, wherein forming the at least one recess involves grinding the guidewire.

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. A guidewire for performing a transseptal puncture, the guidewire comprising:
a distal end and a proximal end with an elongated length therebetween,
the elongated length including at least one recess being located, at least in part, therealong;
the distal end configured to puncture tissue; and,
at least one guidewire marker, wherein the at least one guidewire marker is configured to be securely positioned, respectively at said at least one recess of the elongated length of the guidewire.

2. The guidewire of claim 1, wherein the guidewire has a nominal outer diameter and the at least one guidewire marker remains within the nominal outer diameter of the guidewire.

3. The guidewire of claim 1, wherein the at least one guidewire marker is visually distinct from the guidewire.

4. The guidewire of claim 1, wherein the distal end comprises an energy emitting device.

5. The guidewire of claim 1, wherein the guidewire further comprises an outer layer that is electrically insulating.

6. The guidewire of claim 5, wherein the outer layer encapsulates the at least one guidewire marker.

7. The guidewire of claim 5, wherein the outer layer is a clear, insulating layer.

8. The guidewire of claim 1, wherein the at least one guidewire marker is an inert guidewire marker.

9. The guidewire of claim 8, wherein the inert guidewire marker is composed of a non-inert guidewire marker comprising an inert coating over the non-inert guidewire marker.

10. The guidewire of claim 1, wherein the at least one guidewire marker further comprises an exotic surface configured to promote visibility.

11. The guidewire of claim 10, wherein the exotic surface comprises one or more reflective metallic coils, dimple bands, and knurls.

12. The guidewire of claim 1, wherein the at least one guidewire marker comprises marker contact surfaces and wherein the at least one recess comprises a guidewire contact surface and wherein the guidewire contact surface is configured to securely contact the marker contact surface.

13. The guidewire of claim 1, wherein the at least one recess comprises two or more recesses and wherein the two or more recesses are spaced apart from one another along the elongated length of the guidewire.

14. The guidewire of claim 13, wherein the two or more recesses form a pattern along the elongated length.

15. The guidewire of claim 1, wherein the at least one guidewire marker indicates rotational positioning of the guidewire.

16. The guidewire of claim 1, wherein the at least one guidewire marker mitigates the reduction in stiffness of the at least one recess.

17. The guidewire of claim 1, wherein the at least one guidewire comprises sharp edges.

18. An assembly for performing a transseptal puncture, the assembly comprising:
a guidewire configured for puncturing tissue, the guidewire comprising:
a guidewire distal end and a guidewire proximal end with an elongated length therebetween,
the elongated length including at least one recess being located, at least in part, therealong;
the guidewire distal end configured to puncture tissue; and,
at least one guidewire marker, wherein the at least one guidewire marker is configured to be securely positioned, respectively at said at least one recess of the elongated length of the guidewire; and
an ancillary device comprising an ancillary device distal end and an ancillary device proximal end with a lumen for receiving the guidewire therebetween; and, wherein when the guidewire is inserted within the lumen, the at least one guidewire marker allows the guidewire to be positioned relative to the ancillary device proximal end.

19. The assembly of claim 18, wherein the ancillary device proximal end comprises an ancillary device marker.

20. A method of manufacturing a guidewire comprising at least one recess and at least one guidewire marker, securely contacting said at least one recess, the method of manufacturing comprising the steps of:
forming the at least one recess on the guidewire;
positioning the at least one guidewire marker in the at least one recess;
fixing the at least one guidewire marker in the at least one recess; and
applying an outer layer overtop the guidewire, encapsulating the at least one guidewire marker.
